(19) United States
(12) Patent Application Publication
(10) Pub. No.: US 2022/0387341 A1
(43) Pub. Date: Dec. 8, 2022

(54) PRESSURE-SENSITIVE ADHESIVE MATRIX AND PATCH

(71) Applicant: GUANGDONG HONGSHANHU PHARMACEUTICAL CO., LTD., Dongguan (CN)

(72) Inventors: Gaohua WANG, Dongguan (CN); Yanqiu FANG, Dongguan (CN); Qing WANG, Dongguan (CN); Chunxia XU, Dongguan (CN)

(73) Assignee: GUANGDONG HONGSHANHU PHARMACEUTICAL CO., LTD., Dongguan (CN)

(21) Appl. No.: 17/764,625

(22) PCT Filed: May 21, 2021
(86) PCT No.: PCT/CN2021/095131

§ 371 Date: Mar. 29, 2022

(30) Foreign Application Priority Data
Dec. 28, 2020 (CN) 202011577419.5

(51) Int. Cl. A61K 9/70; A61K 31/426; A61K 31/439; A61K 31/4168; A61K 47/32; A61K 47/12; A61K 47/10

(52) U.S. Cl. CPC A61K 9/7061; A61K 31/426; A61K 31/439; A61K 31/4168; A61K 47/32; A61K 47/12; A61K 47/10

(57) ABSTRACT

A pressure-sensitive adhesive matrix and a patch are disclosed. The pressure-sensitive adhesive matrix includes the following components: in parts by mass, 60-85 parts of an acrylate pressure-sensitive adhesive, 1-20 parts of a polyol, and 5-30 parts of a plasticizer. The pressure-sensitive adhesive matrix has a micro-phase separation structure, and fine polyol droplets are uniformly dispersed in a homogeneous phase structure compounded by the acrylate pressure-sensitive adhesive and plasticizer. The pressure-sensitive adhesive matrix exhibits water absorbability and moisture retention ability, so that the adhesion between the matrix and skin is not adversely influenced by TEWL. The adhesion of the matrix is gradually improved after absorbing water, therefore the adhesion time is increased.

### CROSS REFERENCE TO RELATED APPLICATION

This application claims the priority of Chinese Patent Application No. 202011577419.5, entitled by “Pressure-sensitive adhesive matrix and patch” filed with the Chinese National Intellectual Property Administration on Dec. 28, 2020, the disclosure of which is incorporated by reference herein in its entirety as part of the present application.

### TECHNICAL FIELD

The present disclosure relates to the technical field of pharmaceutical formulations, in particular to a pressure-sensitive adhesive matrix and a patch.

### BACKGROUND ART

Transdermal drug delivery system (TDDS) refers to drug delivery through a skin application method, by which the drug passes through stratum corneum, diffuses through skin, and is absorbed by capillaries into the systemic blood circulation, and an effective plasma concentration is achieved and thereby the purposes of treating and preventing diseases are achieved. Compared with common oral and injection administrations, TDDS has advantages of avoiding gastrointestinal irritation and liver first-pass effect, maintaining a constant plasma concentration, convenience in use and discontinuation. Patch is a common TDDS dosage form, and is composed of a backing layer, a pressure-sensitive adhesive layer, and a release layer. Among them, the pressure-sensitive adhesive plays a role of adhering to skin and transferring drugs to skin, and is an important part of TDDS.

Acrylate pressure-sensitive adhesive is currently one of the most important pressure-sensitive adhesives in the patch market due to its easiness in use and simple preparation process. Acrylate pressure-sensitive adhesive is a hydrophobic high-adhesive polymer. Different types of acrylate pressure-sensitive adhesives exhibit different properties. Some acrylate pressure-sensitive adhesives exhibit a high adhesion to skin and would cause damage to skin when peeled off. Some acrylate pressure-sensitive adhesives exhibit poor moisture permeability, and would fall off due to the adhesion adversely affected by transepidermal water loss (TEWL) after a long-term skin adhesion, which limits its application in TDDS.

### SUMMARY

In view of this, the present disclosure provides a pressure-sensitive adhesive matrix and a patch. The pressure-sensitive adhesive matrix according to the present disclosure has a suitable adhesion property, which allows for no damage to skin when peeled off, and meanwhile ensures the release and permeability of drugs.

In order to achieve the above-mentioned object, the present disclosure provides the following technical solutions:

Disclosed is a pressure-sensitive adhesive matrix, which comprises the following components: in parts by mass, 60-85 parts of an acrylate pressure-sensitive adhesive, 1-20 parts of a polyol, and 5-30 parts of a plasticizer.

In some embodiments, the plasticizer includes at least one selected from the group consisting of a citrate plasticizer, a trimellitate plasticizer, and an epoxy compound plasticizer.

In some embodiments, the citrate plasticizer includes at least one selected from the group consisting of tributyl citrate, trioctyl citrate, tributyl O-acetylcitrate, and tri-n-hexyl O-butyrylcitrate.

The trimellitate plasticizer includes at least one selected from the group consisting of trioctyl trimellitate, trihexyl trimellitate, and triglyceryl trimellitate.

The epoxy compound plasticizer includes at least one selected from the group consisting of epoxy fatty acid butyl ester and epoxy fatty acid octyl ester.

In some embodiments, the polyol includes at least one selected from the group consisting of a diol and a triol.

In some embodiments, the diol is at least one selected from the group consisting of ethylene glycol and propylene glycol, and the triol is glycerol.

In some embodiments, the acrylate pressure-sensitive adhesive includes at least one selected from the group consisting of an acrylate pressure-sensitive adhesive containing a carboxyl group, an acrylate pressure-sensitive adhesive containing a hydroxyl group, and an acrylate pressure-sensitive adhesive containing no functional group.

In some embodiments, the acrylate pressure-sensitive adhesive containing a carboxyl group is at least one selected from the group consisting of duro-tak® 87-2852, duro-tak® 87-2677, duro-tak® 87-2052 and duro-tak® 87-2196;

the acrylate pressure-sensitive adhesive containing a hydroxyl group is at least one selected from the group consisting of duro-tak® 87-2510, duro-tak® 87-2287, duro-tak® 87-2516, duro-tak® 87-2525 and duro-tak® 87-4287;

the acrylate pressure-sensitive adhesive containing no functional group is duro-tak® 87-4098.

In some embodiments, the pressure-sensitive adhesive matrix further includes an antioxidant, and a mass fraction of the antioxidant in the pressure-sensitive adhesive matrix is in the range of 0.05-0.15%.

The present disclosure also provides a patch comprising a drug-loaded pressure-sensitive adhesive layer, and a backing layer and an anti-adhesion layer arranged on surfaces of both sides of the drug-loaded pressure-sensitive adhesive layer respectively. The drug-loaded pressure-sensitive adhesive layer comprises the pressure-sensitive adhesive matrix as described above, a drug, and a permeability enhancer.

In some embodiments, the drug is at least one selected from the group consisting of pramipexole, granisetron and clonidine, and a mass ratio of the drug to the permeability enhancer is in the range of 1:0.5-1.

In some embodiments, the permeability enhancer includes at least one selected from the group consisting of a pyrrolidone permeability enhancer, a fatty acid permeability enhancer, a fatty acid ester permeability enhancer, a fatty alcohol permeability enhancer and a surfactant permeability enhancer.

In some embodiments, the pyrrolidone permeability enhancer includes at least one selected from the group consisting of N-methyl-2-pyrrolidone and 2-pyrrolidone; the fatty acid permeability enhancer includes at least one selected from the group consisting of lauric acid, oleic acid, linoleic acid, and linolenic acid; the fatty acid ester permeability enhancer includes at least one selected from the group consisting of isopropyl myristate, and propylene glycol dipelargonate; the fatty alcohol permeability enhancer includes at least one selected from the group consisting of propylene glycol, octanol and olely alcohol; and the surfactant permeability enhancer includes at least one selected from the group consisting of sodium lauryl sulfate and Tween-80.

In some embodiments, the drug-loaded pressure-sensitive adhesive layer has a thickness of 120 μm.

The present disclosure provides a pressure-sensitive adhesive matrix, which comprises the following components in parts by mass: 60-85 parts of an acrylate pressure-sensitive adhesive, 1-20 parts of a polyol, and 5-30 parts of a plasticizer. The acrylate pressure-sensitive adhesive is a copolymer with a micro-phase separation structure. In the present disclosure, a polyol and a plasticizer are used to modify an acrylate pressure-sensitive adhesive. Fine polyol droplets are uniformly dispersed in a homogeneous-phase structure compounded by an acrylate pressure-sensitive adhesive and a plasticizer, so that the pressure-sensitive adhesive matrix exhibits water absorbability and moisture retention ability, and the water on the body surface is easily absorbed. Thereby, the adhesion between the matrix and skin would not be affected by TEWL. Meanwhile, the adhesion of the matrix after absorbing water and being wetted would gradually increase, which could increase the adhesion time. In addition, the unmodified high-adhesive pressure-sensitive adhesive would cause damage to skin when peeled off, leading to problems such as contact dermatitis, while for the pressure-sensitive adhesive matrix according to the present disclosure, its adhesion performance is controlled to be within an appropriate range because of the synergistic effect of the plasticizer and the polyol (the polyol exhibits a strong hydrophilicity, and could enhance the water absorption of the pressure-sensitive adhesive; the plasticizer could improve elongation, pliability and flexibility of the pressure-sensitive adhesive). Further, in the present disclosure, a plasticizer and a polyol are used to modify the pressure-sensitive adhesive matrix, without adversely affecting the drug release and skin permeability of the matrix. The patch prepared from the pressure-sensitive adhesive matrix of the present disclosure allows for good drug release and skin permeability while maintaining suitable adhesion to skin, and thus is suitable for patients who need a long-term skin administration (such as patients who were developed with Parkinson, Alzheimer, or cardiovascular disease, etc.).

### BRIEF DESCRIPTION OF THE DRAWINGS

FIG. 1 is a graph showing the drug release of the patch prepared in Example 1.

FIG. 2 is a graph showing the drug permeability of the patch prepared in Example 1.

### DETAILED DESCRIPTION OF THE EMBODIMENTS

The present disclosure provides a pressure-sensitive adhesive matrix, which comprises the following components: in parts by mass, 60-85 parts of an acrylate pressure-sensitive adhesive, 1-20 parts of a polyol, and 5-30 parts of a plasticizer.

The pressure-sensitive adhesive matrix according to the present disclosure comprises 60-85 parts by mass, preferably 65-80 parts by mass, more preferably 70-75 parts by mass of the acrylate pressure-sensitive adhesive. In some embodiments of the present disclosure, the acrylate pressure-sensitive adhesive includes at least one selected from the group consisting of an acrylate pressure-sensitive adhesive containing a carboxyl group, an acrylate pressure-sensitive adhesive containing a hydroxyl group, and an acrylate pressure-sensitive adhesive containing no functional group. In some embodiments, the acrylate pressure-sensitive adhesive containing a carboxyl group is at least one selected from the group consisting of duro-tak® 87-2852, duro-tak® 87-2677, duro-tak® 87-2052, and duro-tak® 87-2196. In some embodiments, the acrylate pressure-sensitive adhesive containing a hydroxyl group is at least one selected from the group consisting of duro-tak® 87-2510, duro-tak® 87-2287, duro-tak® 87-2516, duro-tak® 87-2525, and duro-tak® 87-4287. In some embodiments, the acrylate pressure-sensitive adhesive containing no functional group is duro-tak® 87-4098.

In relative to the mass parts of the acrylate pressure-sensitive adhesive, the pressure-sensitive adhesive matrix according to the present disclosure comprises 1-20 parts, preferably 5-15 parts, and more preferably 8-12 parts of the polyol. In some embodiments of the present disclosure, the polyol includes at least one selected from the group consisting of a dihydric alcohol and a trihydric alcohol. In some embodiments, the dihydric alcohol is at least one selected from the group consisting of ethylene glycol and propylene glycol. In some embodiments, the trihydric alcohol is glycerol. In the present disclosure, the hydrophilicity of the pressure-sensitive adhesive matrix could be enhanced by adding the polyol, which is beneficial for the pressure-sensitive adhesive to maintain water absorbabilty and moisture retention ability, and thus the water on the body surface is easily absorbed. Thereby, the adhesion between the matrix and skin would not be affected by TEWL. Meanwhile, the adhesion of the matrix after absorbing water would gradually increase, which could increase the adhesion time.

In relative to the mass parts of the acrylate pressure-sensitive adhesive, the pressure-sensitive adhesive matrix according to the present disclosure comprises 5-30 parts, preferably 10-25 parts, and more preferably 15-20 parts of the plasticizer. In some embodiments of the present disclosure, the plasticizer includes at least one selected from the group consisting of a citrate plasticizer, a trimellitate plasticizer, and an epoxy compound plasticizer. In some embodiments, the citrate plasticizer includes at least one selected from the group consisting of tributyl citrate, trioctyl citrate, tributyl O-acetylcitrate, and tri-n-hexyl O-butyrylcitrate. In some embodiments, the trimellitate plasticizer includes at least one selected from the group consisting of trioctyl trimellitate, trihexyl trimellitate, and glyceryl trimellitate. In some embodiments, the epoxy compound plasticizer includes at least one selected from the group consisting of epoxy butyl fatty acid ester and epoxy octyl fatty acid ester. In the present disclosure, by adding the plasticizer, the plasticity of the pressure-sensitive adhesive could be improved, accompanying with an enhanced elongation, pliability and flexibility, which facilitates the attachment and adhering of the pressure-sensitive adhesive, and improves the coating conditions of the pressure-sensitive adhesive.

In some embodiments of the present disclosure, the pressure-sensitive adhesive matrix further includes an antioxidant. In some embodiments, a mass fraction of the antioxidant in the pressure-sensitive adhesive matrix is in the range of 0.05-0.15%, preferably 0.1%. There is no special requirement on the type of the antioxidant in the present disclosure, and antioxidants well known to those skilled in the art could be used.

The present disclosure also provides a patch comprising a drug-loaded pressure-sensitive adhesive layer, and a backing layer and an anti-adhesion layer arranged on surfaces of both sides of the drug-loaded pressure-sensitive adhesive layer respectively. The drug-loaded pressure-sensitive adhesive layer includes the pressure-sensitive adhesive matrix as described above, a drug, and a permeability enhancer.

In some embodiments of the present disclosure, the drug is at least one of pramipexole, granisetron, and clonidine. There is no special requirement on the drug loading amount in the drug-loaded pressure-sensitive adhesive layer. In some embodiments, the drug loading amount is determined according to the type of drug. In a specific embodiment of the present disclosure, a drug loading amount in the drug-loaded pressure-sensitive adhesive layer is in the range of 10%. In some embodiments of the present disclosure, the permeability enhancer includes at least one selected from the group consisting of a pyrrolidone permeability enhancer, a fatty acid permeability enhancer, a fatty acid ester permeability enhancer, a fatty alcohol permeability enhancer, and a surfactant permeability enhancer. In some embodiments, the pyrrolidone permeability enhancer includes at least one selected from the group consisting of N-methyl-2-pyrrolidone and 2-pyrrolidone. In some embodiments, the fatty acid permeability enhancer includes at least one selected from the group consisting of lauric acid, oleic acid, linoleic acid, and linolenic acid. In some embodiments, the fatty acid ester permeability enhancer includes at least one selected from the group consisting of isopropyl myristate and propylene glycol dipelargonate. In some embodiments, the fatty alcohol permeability enhancer includes at least one selected from the group consisting of propylene glycol, octanol and olely alcohol. In some embodiments, the surfactant permeability enhancer includes at least one selected from the group consisting of sodium lauryl sulfate and Tween-80. In some embodiments, a mass ratio of the drug to the permeability enhancer is in the range of 1:0.5-1, and preferably 1:0.6-0.8.

In some embodiments of the present disclosure, the drug-loaded pressure-sensitive adhesive layer has a thickness of 120 μm.

There is no special requirement on the backing layer and the anti-adhesion layer in the present disclosure, and the backing layer and the anti-adhesion layer that are well known to those skilled in the art could be used.

In a specific embodiment of the present disclosure, the time for a stable adhesion between the patch and skin is 48 hours or more.

In some embodiments of the present disclosure, the method for preparing the patch includes the following steps:

stirring and mixing components of the drug-loaded pressure-sensitive adhesive layer, coating the resulting mixture on a backing layer and drying to obtain a drug-loaded pressure-sensitive adhesive layer, and finally arranging an anti-adhesion layer on a surface of the drug-loaded pressure-sensitive adhesive layer.

In some embodiments of the present disclosure, the stirring and mixing is performed at room temperature. In some embodiments, the stirring and mixing is performed for 90 minutes.

There is no special requirement for the specific conditions for coating and drying in the present disclosure, and conditions well known to those skilled in the art may be used.

The technical solutions of the present disclosure will be clearly and completely described below in conjunction with the examples of the present disclosure.

Test methods for adhesion, peeling strength, drug release performance and drug permeability performance of the patch in the examples were as follows.

The adhesion test was carried out in accordance with the method in the national standard (GB/T 4851-1998). Under a condition of 23±2° C. and a relative humidity of 65±5%, a pressure-sensitive tape to be test with a length of 70 mm and a width of 25 mm was adhered to two adjacent test boards (one of which is used as a loading board), such that the adhering length L was 25 mm, and the adhering length on the loading board is twice greater than L. The above-prepared test piece was kept at room temperature for 2 hours or more, and hung vertically on the test rack with the test temperature, then a weigh of 1000 g was vertically hung at the lower end of the loading board and at the same time the timing started. The time when the patch fell off the test boards was recorded. The measurement was repeated 3 times for one sample, and the adhesion was evaluated by the time when the patch fell off the test boards.

The peeling strength test was carried out in accordance with the method in the national standard (GB 2792-81). Under a condition of 23±2° C. and a relative humidity of 65±5%, a patch with a length of 100 mm and a width of 25 mm was adhered to a test board and then kept for 2 hours or more. A 180° peeling experiment was performed at a set peeling speed. The maximum, minimum and average values were recorded. The measurement was repeated 3 times for one sample.

The test method for drug release performance was as follows. The drug release performance of patch was determined by a paddle disc method described in the fourth method in general rule 0931, Part IV of Chinese Pharmacopoeia, 2015 edition. A patch was taken, the anti-adhesive layer thereof was peeled off, the adhesive layer thereof was adhered upward to a stainless steel mesh dish, and then the patch was placed in a dissolution vessel containing 900 mL of purified water at a temperature of 32±0.5° C. and a stirring speed of 50 r/min. A sample of 5 mL in the dissolution vessel was taken at 1 h, 12 h and 24 h respectively (and an equal amount of blank medium with the same temperature was supplemented at the same time). The concentration of each active ingredient was determined by HPLC. The cumulative release rate Q₁ is calculated according to formulas (1) and (2).

$$M t = \frac{{C}_{n} \times V + \sum {C}_{n - 1} \times 5}{A} \qquad formula ( 1 )$$

$${Q}_{1} = \frac{{M}_{t}}{{M}_{∞}} \times 100 \% \qquad formula ( 2 )$$

where Q₁ represents a cumulative release rate at the time t; Mₜ represents a cumulative release amount per unit area; M_{∞}represents a drug loading amount per unit area of patch; V represents a received medium volume; A represents a release area; and Cₙ, represents a concentration at the nth sampling.

The method for determining drug permeability was as follows. A fresh human skin was used, and the dermis layer was faced to a receiving pool. A patch was cut to have a size of a diffusion area, and the protective film of the patch was removed, then the patch was adhered to the surface of the skin stratum corneum. The diffusion area was 0.627 cm², and the receiving pool had a capacity of 5 mL, and contained a medium of pure water with a temperature of 37±0.5° C., and the stirring speed therein was 700 r/min. A sample of 0.4 mL was taken from the receiving pool at 1 h, 6 h, 12 h, 18 h, 24 h and 48 h respectively (and an equal amount of blank medium with the same temperature was supplemented at the same time). The concentration of each active ingredient was determined by HPLC. The cumulative permeation amount Q₂ is calculated according to formula (3).

$${Q}_{2} = \frac{{C}_{n} \times V + \sum {C}_{n - 1} \times {V}_{n}}{A} \qquad formula ( 3 )$$

where Q₂ represents a cumulative permeation amount at the time t; Vₙ represents a sampling volume; A represents an effective permeation area; and Cₙ represents a concentration at the nth sampling.

### Example 1

The components of a drug-loaded pressure-sensitive adhesive matrix were as follows: in parts by mass, 60 parts of acrylate pressure-sensitive adhesive duro-tak® 87-4098, 30 parts of trioctyl trimellitate, 1 part of propylene glycol, 5 parts of pramipexole, and 4 parts of isopropyl myristate.

The above components were added into a stirring tank, stirred for 90 minutes at room temperature. The resulting material was then discharged, applied onto a backing layer and dried, forming a patch with a drug-loaded pressure-sensitive adhesive layer of 120 μm in thickness. For the convenience of testing, a test was performed directly after the drug-loaded pressure-sensitive adhesive layer was formed, without setting an anti-adhesion layer.

### Example 2

This example was the same as Example 1, except that the components of the drug-loaded pressure-sensitive adhesive matrix were as follows: in parts by mass, 60 parts of acrylate pressure-sensitive adhesive duro-tak® 87-2677, 10 parts of glyceryl trimellitate, 20 parts of propylene glycol, 5 parts of pramipexole, and 5 parts of isopropyl myristate.

### Example 3

This example was the same as Example 1, except that the components of the drug-loaded pressure-sensitive adhesive matrix were as follows: in parts by mass, 85 parts of acrylate pressure-sensitive adhesive duro-tak® 87-2510, 5 parts of tributyl citrate, 1 part of glycerol, 5 parts of pramipexole, and 4 parts of isopropyl myristate.

### Example 4

This example was the same as Example 1, except that the components of the drug-loaded pressure-sensitive adhesive matrix were as follows: in parts by mass, 85 parts of acrylate pressure-sensitive adhesive duro-tak® 87-2287, 5 parts of triethyl citrate, 1 part of glycerol, 5 parts of pramipexole, and 4 parts of isopropyl myristate.

### Example 5

This example was the same as Example 1, except that the components of the drug-loaded pressure-sensitive adhesive matrix were as follows: in parts by mass, 65 parts of acrylate pressure-sensitive adhesive duro-tak® 87-2287, 20 parts of tri-n-hexyl O-butyrylcitrate, 5 parts of glycerol, 5 parts of pramipexole, and 5 parts of isopropyl myristate.

### Example 6

This example was the same as Example 1, except that the components of the drug-loaded pressure-sensitive adhesive matrix were as follows: in parts by mass, 70 parts of an acrylate pressure-sensitive adhesive, 10 parts of epoxy butyl fatty acid ester, 10 parts of glycerol, 5 parts of pramipexole, and 5 parts of isopropyl myristate, wherein a mixture of duro-tak® 87-2677 and duro-tak® 87-2516 (in a mass ratio of 1:1) was used as the acrylate pressure-sensitive adhesive.

### Example 7

This example was the same as Example 1, except that the components of the drug-loaded pressure-sensitive adhesive matrix were as follows: in parts by mass, 60 parts of acrylate pressure-sensitive adhesive, 30 parts of trioctyl citrate, 1 part of glycerol, 5 parts of pramipexole, and 4 parts of isopropyl myristate, wherein a mixture of duro-tak® 87-2852 and duro-tak® 87-2287 (in a mass ratio of 1:1) was used as the acrylate pressure-sensitive adhesive.

### Example 8

This example was the same as Example 1, except that the components of the drug-loaded pressure-sensitive adhesive matrix were as follows: in parts by mass, 80 parts of an acrylate pressure-sensitive adhesive, 1 part of epoxy octyl fatty acid ester, 10 parts of glycerol, 5 parts of pramipexole, and 4 parts of isopropyl myristate, wherein a mixture of duro-tak® 87-2052 and duro-tak® 87-4098 (in a mass ratio of 1:1) was used as the acrylate pressure-sensitive adhesive.

### Comparative Example 1

This example was the same as Example 1, except that the components of the drug-loaded pressure-sensitive adhesive matrix were as follows: in parts by mass, 85 parts of acrylate pressure-sensitive adhesive duro-tak® 87-2677, 5 parts of pramipexole, and 10 parts of isopropyl myristate.

### Comparative Example 2

This example was the same as Example 1, except that the components of the drug-loaded pressure-sensitive adhesive matrix were as follows: in parts by mass, 75 parts of an acrylate pressure-sensitive adhesive, 5 parts of pramipexole, and 20 parts of isopropyl myristate, wherein a mixture of duro-tak® 87-2677 and duro-tak® 87-2516 (in a mass ratio of 1:1) was used as the acrylate pressure-sensitive adhesive.

Adhesion Performance Test

The peeling strength and adhering time of the patches prepared in Examples 1 to 8 and Comparative Examples 1 to 2 were tested. The results are listed in Table 1.

TABLE 1
Results of adhesion performance test

| | 180° peeling | Adhering |
|---|---|---|
| Comparative Examples/Examples | strength/KN/m | time/h |
| Comparative Example 1 | 2 | >24 |
| Comparative Example 2 | 0.02 | 0.5-0.8 |
| Example 1 | 0.1-0.25 | 1-1.5 |
| Example 2 | 0.1-0.30 | 1.2-1.5 |
| Example 3 | 0.15-0.40 | 4-10 |
| Example 4 | 0.30-0.50 | 3-7 |
| Example 5 | 0.20-0.30 | 1.5-2.5 |
| Example 6 | 0.20-0.50 | 5-7 |
| Example 7 | 0.40-0.60 | 0.8-1.8 |
| Example 8 | 0.20-0.40 | 3-5 |

In Table 1, the peeling strengths and adhering times of Examples 1 to 8 are the value ranges formed by the minimum value and the maximum value of three parallel tests; the peel strengths of Comparative Examples 1 to 2 are the average values of three parallel tests; the adhering time of Comparative Example 2 is the value range formed by the minimum value and the maximum value of three parallel tests.

According to the results in Table 1, in Comparative Example 1, the acrylate pressure-sensitive adhesive used is duro-tak® 87-2677, which exhibits a good bonding property and a long adhering time, but exhibits an excessively high peeling strength, thereby causing damage to human skin when the patch is peeled off. In Example 2, the acrylate pressure-sensitive adhesive used is the same type as that in Comparative Example 1; after the pressure-sensitive adhesive matrix is modified with the plasticizer and polyol, the patch exhibits a peeling strength that is controlled within the range of 0.1-0.3 KN/m, and an adhering time of 1.2-1.5 h, which could meet the adhesion requirements and would not cause damage to the skin when the patch is peeled off. In Comparative Example 2, the acrylate pressure-sensitive adhesive used is duro-tak® 87-2677 and duro-tak® 87-2516, and the latter pressure-sensitive adhesive exhibits a poor moisture permeability, a poor adhesion and a short adhering time. In Example 6, the acrylate pressure-sensitive adhesive used is the same as that in Comparative Example 2; after the pressure-sensitive adhesive matrix is modified according to the present disclosure, the obtained patch exhibits an adhering time of 5-7 h, and a peeling strength that is controlled within the range of 0.20-0.50 KN/m, so that the patch would not cause damage to the human body when peeled off. According to the test results of other examples, it can be seen that the adhesion performance of the patch could be controlled within a suitable range by compounding the acrylate pressure-sensitive adhesive with a plasticizer and a polyol according to the present disclosure, which meets the requirements on the adhering time and meanwhile does not cause damage to human skin.

The adhesion performance tests of the patches prepared in Examples 1 to 8 on human skin were performed. The results show that the obtained patches could adhere to human skin stably for 48 hours or more, and would not cause damage to human skin when peeled off.

Water Absorption Experiment

The water absorption experiment was carried out on the patch prepared in Example 1. The experimental procedure was as follows. The patch prepared in Example 1 was taken and placed in a test box at a temperature of 25±1° C. and a relative humidity of 80±2%. After 24 hours, the adhering time and peeling strength of the patch were measured. The results show that the patch prepared in Example 1 after absorbing exhibits an adhering time increased by 3 times and a peeling strength increased by 8 times. This shows that the pressure-sensitive adhesive matrix of the present disclosure exhibit good water absorbability, which is conductive to increasing adhesion performance. In practical applications, the adhesion between the matrix and skin would not be adversely affected by TEWL.

Tests for Drug Release Performance and Drug Permeability Performance

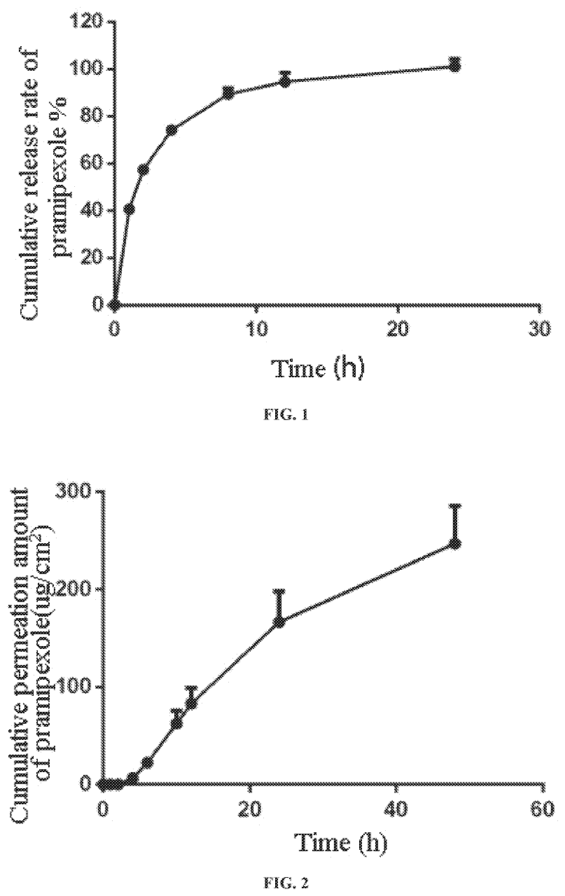

The drug release performance and drug permeability performance of the patch prepared in Example 1 were tested. The results are shown in FIGS. 1 and 2. FIG. 1 shows the drug release curve of the patch of Example 1, and FIG. 2 shows the drug permeability of the patch of Example 1. According to FIG. 1, it can be seen that after 24 h, the release rate of the drug in the patch could reach 100%. According to FIG. 2, it can be seen that after 48 h, the permeation amount of the drug in the patch into the skin could reach 250 μg/cm².

The drug release performance and drug permeability performance of the patches prepared in Examples 2-8 were tested. The results are similar to those of Example 1, indicating that the obtained patches exhibit good drug release performance and drug permeability performance.

The drug release performance and drug permeability performance of the patches prepared in Comparative Examples 1-2 were tested. The results show that in comparison with Comparative Examples 1-2, in Example 1, the 24-hour cumulative drug release rate of the patch is slightly improved, and the skin permeability is not significantly changed. This result shows that the modification of the acrylate pressure-sensitive adhesive matrix with the plasticizer and polyol would not adversely affect the drug release performance and skin permeability performance of the matrix.

The present disclosure is supported by DongGuan Innovative Research Team Program, China.

The foregoing descriptions are merely preferred embodiments of the present disclosure. It should be noted that those of ordinary skill in the art may make a number of improvements or modifications without departing from the principle of the present disclosure. These improvements or modifications should also fall within the scope of the present disclosure.

1. A pressure-sensitive adhesive matrix, comprising, in parts by mass, 60-85 parts of an acrylate pressure-sensitive adhesive, 1-20 parts of a polyol, and 5-30 parts of a plasticizer.

2. The pressure-sensitive adhesive matrix of claim 1, wherein the plasticizer comprises at least one selected from the group consisting of a citrate plasticizer, a trimellitate plasticizer, and an epoxy compound plasticizer.

3. The pressure-sensitive adhesive matrix of claim 2, wherein the citrate plasticizer comprises at least one selected from the group consisting of tributyl citrate, trioctyl citrate, tributyl O-acetylcitrate, and tri-n-hexyl O-butyrylcitrate;
the trimellitate plasticizer comprises at least one selected from the group consisting of trioctyl trimellitate, trihexyl trimellitate, and glyceryl trimellitate; and
the epoxy compound plasticizer comprises at least one selected from the group consisting of epoxy fatty acid butyl ester and epoxy fatty acid octyl ester.

4. The pressure-sensitive adhesive matrix of claim 1, wherein the polyol comprises at least one selected from the group consisting of a diol and a triol.

5. The pressure-sensitive adhesive matrix of claim 4, wherein the diol is at least one selected from the group consisting of ethylene glycol and propylene glycol, and the triol is glycerol.

6. The pressure-sensitive adhesive matrix of claim 1, wherein the acrylate pressure-sensitive adhesive comprises at least one selected from the group consisting of an acrylate pressure-sensitive adhesive containing a carboxyl group, an acrylate pressure-sensitive adhesive containing a hydroxyl group, and an acrylate pressure-sensitive adhesive containing no functional group.

7. The pressure-sensitive adhesive matrix of claim 6, wherein the acrylate pressure-sensitive adhesive containing a carboxyl group is at least one selected from the group consisting of duro-tak® 87-2852, duro-tak® 87-2677, duro-tak® 87-2052, and duro-tak® 87-2196;
the acrylate pressure-sensitive adhesive containing a hydroxyl group is at least one selected from the group consisting of duro-tak® 87-2510, duro-tak® 87-2287, duro-tak® 87-2516, duro-tak® 87-2525, and duro-tak® 87-4287; and
the acrylate pressure-sensitive adhesive containing no functional group is duro-tak® 87-4098.

8. The pressure-sensitive adhesive matrix of claim 1, further comprising an antioxidant, wherein a mass fraction of the antioxidant in the pressure-sensitive adhesive matrix is in the range of 0.05-0.15%.

9. A patch, comprising a drug-loaded pressure-sensitive adhesive layer, and a backing layer and an anti-adhesion layer arranged on surfaces of both sides of the drug-loaded pressure-sensitive adhesive layer respectively, wherein the drug-loaded pressure-sensitive adhesive layer comprises a pressure-sensitive adhesive matrix, a drug, and a permeability enhancer, the pressure-sensitive adhesive matrix comprising, in parts by mass, 60-85 parts of an acrylate pressure-sensitive adhesive, 1-20 parts of a polyol, and 5-30 parts of a plasticizer.

10. The patch of claim 9, wherein the drug is at least one selected from the group consisting of pramipexole, granisetron, and clonidine, and a mass ratio of the drug to the permeability enhancer is in the range of 1:0.5-1.

11. The patch of claim 9, wherein the permeability enhancer comprises at least one selected from the group consisting of a pyrrolidone permeability enhancer, a fatty acid permeability enhancer, a fatty acid ester permeability enhancer, a fatty alcohol permeability enhancer, and a surfactant permeability enhancer.

12. The patch of claim 11, wherein the pyrrolidone permeability enhancer comprises at least one selected from the group consisting of N-methyl-2-pyrrolidone and 2-pyrrolidone;
the fatty acid permeability enhancer comprises at least one selected from the group consisting of lauric acid, oleic acid, linoleic acid and linolenic acid;
the fatty acid ester permeability enhancer comprises at least one selected from the group consisting of isopropyl myristate and propylene glycol dipelargonate;
the fatty alcohol permeability enhancer comprises at least one selected from the group consisting of propylene glycol, octanol, and olely alcohol; and
the surfactant permeability enhancer comprises at least one selected from the group consisting of sodium lauryl sulfate and Tween-80.

13. The patch of claim 9, wherein the drug-loaded pressure-sensitive adhesive layer has a thickness of 120 μm.

14. The patch of claim 10, wherein the permeability enhancer comprises at least one selected from the group consisting of a pyrrolidone permeability enhancer, a fatty acid permeability enhancer, a fatty acid ester permeability enhancer, a fatty alcohol permeability enhancer, and a surfactant permeability enhancer.

15. The patch of claim 9, wherein the plasticizer comprises at least one selected from the group consisting of a citrate plasticizer, a trimellitate plasticizer, and an epoxy compound plasticizer.

16. The patch of claim 15, wherein the citrate plasticizer comprises at least one selected from the group consisting of tributyl citrate, trioctyl citrate, tributyl O-acetylcitrate, and tri-n-hexyl O-butyrylcitrate;
the trimellitate plasticizer comprises at least one selected from the group consisting of trioctyl trimellitate, trihexyl trimellitate, and glyceryl trimellitate; and
the epoxy compound plasticizer comprises at least one selected from the group consisting of epoxy fatty acid butyl ester and epoxy fatty acid octyl ester.

17. The patch of claim 9, wherein the polyol comprises at least one selected from the group consisting of a diol and a triol.

18. The patch of claim 17, wherein the diol is at least one selected from the group consisting of ethylene glycol and propylene glycol, and the triol is glycerol.

19. The patch of claim 9, wherein the acrylate pressure-sensitive adhesive comprises at least one selected from the group consisting of an acrylate pressure-sensitive adhesive containing a carboxyl group, an acrylate pressure-sensitive adhesive containing a hydroxyl group, and an acrylate pressure-sensitive adhesive containing no functional group.

20. The patch of claim 9, wherein the pressure-sensitive adhesive matrix further comprises an antioxidant, wherein a mass fraction of the antioxidant in the pressure-sensitive adhesive matrix is in the range of 0.05-0.15%.
